(19) United States
(12) Patent Application Publication
(10) Pub. No.: US 2009/0105133 A1
(43) Pub. Date: Apr. 23, 2009

(54) Gamma-Delta T Cell Receptors

(75) Inventor: Jonathan Michael Boulter, Cardiff (GB)

Correspondence Address: BANNER & WITCOFF, LTD., 1100 13th STREET, N.W., SUITE 1200, WASHINGTON, DC, 20005-4051, US

(73) Assignee: MEDIGENE LIMITED, Oxfordshire (GB)

(21) Appl. No.: 11/667,863

(22) PCT Filed: Oct. 31, 2005
(86) PCT No.: PCT/GB2005/004198

§ 371 Date: Nov. 12, 2008

(30) Foreign Application Priority Data
Nov. 23, 2004 (GB) 0425732.5

(51) Int. Cl. A61K 38/17; C07K 14/725; C12N 5/06; C12N 15/12; C12P 21/00; C12N 5/10; C12N 15/63; A61P 35/00; G01N 33/53

(52) U.S. Cl. 514/12; 530/300; 435/325; 435/7.21; 536/23.5; 435/320.1; 435/69.1; 435/348

(57) ABSTRACT

The present invention provides gamma-delta T cell receptors (γδTCRs) with an introduced disulfide interchain bond. Such proteins, and cells expressing of such proteins on the surface thereof, have value in methods for distinguishing between cell populations by the TCR ligand they present, and in the treatment of diseases.

The present invention relates to gamma-delta T cell receptors (γδ TCRs) with an introduced disulfide interchain bond. Such proteins and cells expressing of such proteins on the surface thereof have value in methods for distinguishing between cell populations by the γδ TCR ligand they present, and in the treatment of diseases.

### BACKGROUND TO THE INVENTION

### Soluble γδ TCRs

U.S. Pat. No. 5,601,822 discloses soluble polypeptides comprising at least a fragment of a TCR γ or TCR δ chain and antibodies which recognise such polypeptides. Also disclosed are methods of isolating γδ TCRs from γδ T cells.

U.S. Pat. No. 5,185,250 discloses methods for identifying and isolating DNA encoding γδ TCRs and the use of such DNA in cloning methods for causing cells to produce γδ TCRs. Also disclosed are methods of producing antibodies which recognise γδ TCRs.

US 2003/0175212 details the production of soluble γδ TCRs and the use thereof in the treatment of Listeriosis. These soluble γδ TCRs were produced using a baculoviral system in High Five insect cells. The γδ TCRs were truncated so that only the extracellular portions of the TCR chains were secreted, the TCR chains preferably included the native disulfide interchain bond.

In WO 99/60120, a soluble TCR is described which is correctly folded so that it is capable of recognising its native ligand, is stable over a period of time, and can be produced in reasonable quantities. This TCR comprises a TCR α or γ chain extracellular domain dimerised to a TCR β or δ chain extracellular domain respectively, by means of a pair of C-terminal dimerisation peptides, such as leucine zippers. This strategy of producing TCRs is generally applicable to all TCRs.

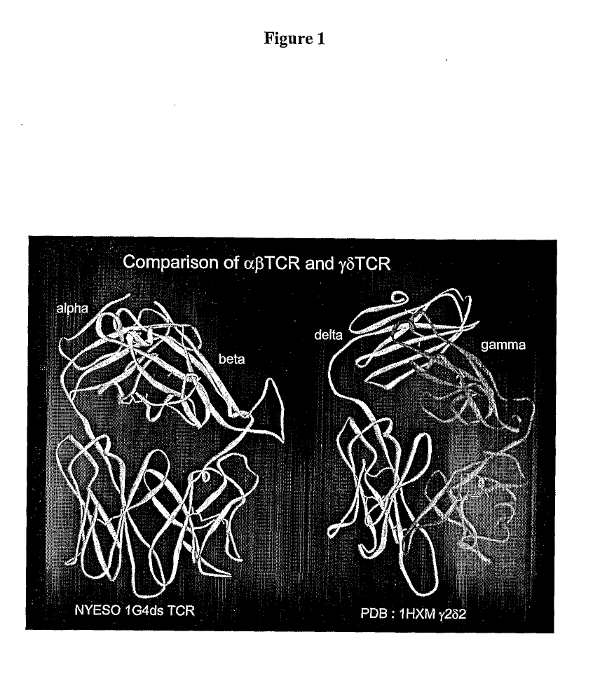

The tertiary structure of up TCRs and γδ TCRs are to some extent similar, with the α TCR chain most closely resembling the δ TCR chain, and the β TCR chain most closely resembling the γ TCR chain. For example, the constant region of the γ TCR chain contains an unpaired cysteine residue as does the constant region of the β TCR chain. However, the tertiary structures of αβ TCRs and γδ TCRs show considerable differences. For example, as illustrated in FIG. 1 there are considerable differences between the tertiary structure of the γ and δ TCR constant domains when compared to the β and α constant domains respectively. There are also considerable differences between the tertiary structures of the γ variable regions compared to the β variable regions. Crystal structure analysis of the δ TCR variable domain revealed that the framework structure of this domain more closely resembled that of the variable immunoglobulin heavy chain domain (V_{H}) that TCR Vα or Vβ domains. (Kabelitz et al., (2000) Int Arch Allergy Immunol 2000 122 1-7)

WO 03/020763 relates to methods for the production of soluble αβ dimeric TCRs (dTCRs) characterised by the presence of a disulfide inter-chain bond between constant domain residues which is not present in native TCR. It discloses specific positions in the dTCRs at which said disulfide bond can be introduced. One preferred site for the introduction of the disulphide bond which is not present in native TCR is between cysteine residues substituted for Thr 48 of exon 1 of TRAC*01 and Ser 57 of exon 1 of TRBC1*01 or TRBC2*01. (IMGT nomenclature as described in (LeFranc et al, (2001) The T cell receptor Factsbook, Academic Press). WO 2004/033685 relates to single-chain up TCRs (scTCRs) containing the introduced disulfide interchain bonds as in WO 03/020763 and further comprising a peptide linker sequence between the C-terminus of one of the TCRs and the N-terminus of the other TCR chain.

(Guillaume et al, 2003 Nature Immunology 4 657-663) details the construction of a soluble JM22 TCR containing an introduced disulfide inter-chain bond between amino acids attached to the C terminus of the construct. This particular construct was derived from the extracellular portion of the JM22 TCR, truncated a single amino acid N terminal to the position of the native disulfide inter-chain bond. C terminal constant domain extensions were added to both the α and β chains of this TCR. These extensions caused the position of the inter-chain forming cysteine residues to be displaced downstream by three amino acids in the α chain and six amino acids in the β chain relative to their native positions. WO 2004048410 provides further details of such TCR constructs.

Surprisingly, in light of the structural disparity between αβ TCRs and γδ TCRs noted above, it has been discovered that soluble γδ TCRs can be stabilised via the introduction of a novel disulfide interchain bond. The γδ TCRs of the present invention are provided in forms similar to the single chain TCRs (scTCRs) or heterodimeric TCRs (dTCRs) described in WO 04/033685 and WO 03/020763 respectively, or in forms comprising introduced C terminal constant domain extensions containing a disulfide inter-chain disulfide bond.

### BRIEF DESCRIPTION OF THE INVENTION

This invention makes available for the first time a γδ TCR, which comprises all or part of a TCR γ chain, and all or part of a TCR δ chain, wherein the TCR chains are linked by a disulfide bond which is not present in native γδ TCRs.

### DETAILED DESCRIPTION OF THE INVENTION

The present invention provides a γδ TCR, which comprises all or part of a TCR γ chain, and all or part of a TCR δ chain, wherein the TCR chains are linked by a disulfide bond which is not present in native γδ TCRs.

The γδ TCRs disclosed herein are useful, inter alia as targeting moieties. Evidence suggests that they target ligands including, but not limited to, small phosphorylated non-pepidic compounds such as isopentenyl pyrophosphate (IPP) which is a constituent of the mevalonate metabolic pathway. (Gober et al., (2003) J Exp Med 197 (2) 163-168) Two further studies (Kato et al., (2003) J. Immunol 170 3608-3613) and (Green et al., (2004) Clin Exp Immunol (2004) 136 472-482) demonstrate that human Vγ9 Vδ2 T cells only respond to IPP when it is presented on the surface of a human cell. (Green et al., (2004) Clin Exp Immunol (2004) 136 472-482) demonstrated that nine different human cell lines, including those deficient in β2-microglobulin and MHC Class I expression, were capable of presenting IPP to Vγ9 Vδ2 T cells. However, the identity of the IPP-presenting complex remains elusive. The results in (Gober et al., (2003) J Exp Med 197 (2) 163-168) and (Green et al., (2004) Clin Exp Immunol (2004) 136 472-482) also provide a plausible explanation for the activation of Vγ9 Vβ2 T cells by certain aminobisphosphonates (nBPs) such as risedronate, zoledronate and pamidronate. These nBPs were considered to be putative ligands for γδ TCRs, however it now seems probable that they promote a γδ T cell response by inhibiting with an enzyme (Farnesyl pyrophosphate (FPP)-synthase) involved in the mevalonate metabolic pathway. Inhibition of this enzyme leads to a build up of IPP which, as stated previously, is now considered to be a γδ TCR ligand.

The expression of hydroxy-methylglutaryl-CoA reductase, the rate-limiting enzyme in the mevalonate metabolic pathway is up-regulated in at least some haematological malignancies (Harwood et al., (1991) J. Lipid Res 32 1237-1252) and mammary carcinoma cells (Asslan et al, Biochem Biophys Res Comm (1999) 260 699-706) This leads to a built up of IPP in these cells, which in turn renders them susceptible to attack by Vγ9 Vδ2 T cells. (Gober et al., (2003) J Exp Med 197 (2) 163-168) lists the non-Hodgkin B cell lymphomas line Daudi, B cell lymphoma RPMI-8233, T cell lymphoma MOLT-4 and the erythroleukemia line K562 as being susceptible to attack by Vγ9 Vδ2 T cells.

γδ T cells are present in organised lymphoid tissues as well as in the skin- and gut-associated lymphoid systems without any special tropism for epithelia. (Gober et al., (2003) J Exp Med 197 (2) 163-168) The sub-populations of human γδ T cells that occur vary in their variable gene usage dependent of their location in the body such as within the gut, brain, blood etc. For example, Vγ3Vδ1 presenting γδ T cells are the dominant sub-population in the gut. This tissue-specificity of different γδ T cells and therefore γδ TCRs makes them useful for the tissue-specific delivery of therapeutic and diagnostics agents. Furthermore, the ligands recognised by this tissue-specific γδ T cells are also useful potential targets for non-TCR-based targeting agents such as antibody-based targeting agents.

According to a further aspect, the present invention provides a soluble T cell receptor (sTCR), which comprises (i) all or part of a TCR γ chain, except the transmembrane domain thereof, and (ii) all or part of a TCR δ chain, except the transmembrane domain thereof, wherein (i) and (ii) each comprise a functional variable domain and at least a part of the constant domain of the TCR chain, and are linked by a disulphide bond which is not present in native TCR.

The γδ TCRs of the invention may be in the form of either single chain TCRs (scTCRs) or heterodimeric TCRs (dTCRs), in particular:

A suitable soluble γδ TCR comprises all or part of a TCR γ chain except the transmembrane domain thereof and all or part of a TCR δ chain except the transmembrane domain thereof, wherein each TCR chain comprise a functional variable domain and at least a part of the constant domain of the TCR chain and are linked by a disulfide bond between constant domain residues which is not present in native TCR.

In one specific embodiment of the invention such γδ TCRs comprise all of the extracellular constant Ig domain of the TCR chain.

In another specific embodiment of the invention such γδ TCRs comprise all of the extracellular domain of the TCR chain.

The γδ TCRs are characterised by having a disulfide link between constant domain residues which is not present in native TCR.

In one aspect of the invention this covalent disulfide bond links a residue of the immunoglobulin region of the constant domain of the γ chain to a residue of the immunoglobulin region of the constant domain of the δ chain.

Another aspect of the invention is provided wherein, in the γδ TCR, an interchain disulfide bond present in native TCRs is absent. A specific embodiment of this aspect is provided wherein, in the soluble γδ TCR, native γ and δ TCR chains are truncated at the C-terminus such that the cysteine residues which form the native interchain disulfide bond are excluded. In an alternative embodiment the cysteine residues which form the native interchain disulfide bond are substituted to another residue. In another specific embodiment, the cysteine residues which form the native interchain disulfide bond are substituted to serine or alanine.

Another aspect of the present invention is provided wherein, in the γδ TCR, the disulfide bond which is not present in native TCRs is between cysteine residues substituted for residues whose β carbon atoms are less than 0.6 nm apart in the native TCR structure.

ImmunoGeneTics (IMGT) nomenclature as described in (LeFranc et al, (2001) The T cell receptor Factsbook, Academic Press) will be used throughout this application to denote the position of particular amino acid residues in TCR chains.

A specific embodiment of the invention is provided wherein, in the γδ TCR, the disulfide bond which is not present in native TCRs is between cysteine residues substituted for Gly 60 of exon 1 of TRGC1 or TRGC2(2×) or TRGC2(3×) and Ala 47 of exon 1 of TRDC.

A specific embodiment of the invention is provided wherein, in the γδ TCR, the disulfide bond which is not present in native TCRs is between cysteine residues substituted for residue 60 of exon 1 of TRGC1 and residue 47 of exon 1 of TRDC.

A specific embodiment of the invention is provided wherein, in the γδ TCR, the disulfide bond which is not present in native TCRs is between cysteine residues substituted for residue 49 of TRGV and a residue of TRDJ located in a motif FGXG (SEQ ID NO: 1) wherein X denotes the residue to be substituted

A specific embodiment of the invention is provided wherein, in the γδ TCR, the disulfide bond which is not present in native TCRs is between cysteine residues substituted for Val 49 of TRGV9 and a residue of TRDJ located in a motif FGXG (SEQ ID NO: 1) wherein X denotes the residue to be substituted

A specific embodiment of the invention is provided wherein, in the γδ TCR, the disulfide bond which is not present in native TCRs is between cysteine residues substituted for residue 49 of TRGV and a residue of TRDJ1 located in a motif FGXG (SEQ ID NO: 1) wherein X denotes the residue to be substituted

A specific embodiment of the invention is provided wherein, in the γδ TCR, the disulfide bond which is not present in native TCRs is between cysteine residues substituted for a residue of TRGJ located in the following motif:

| | |
|---|---|
| FX₁X₂G | (SEQ ID NO: 2) |

Wherein X₂ denotes the residue to be substituted

And residue 49 of TRDV.

A specific embodiment of the invention is provided wherein, in the γδ TCR, the disulfide bond which is not present in native TCRs is between cysteine residues substituted for a residue of TRGJP located in the following motif:

| | |
|---|---|
| FX₁X₂G | (SEQ ID NO: 2) |

Wherein X₂ denotes the residue to be substituted

And residue 49 of TRDV.

A specific embodiment of the invention is provided wherein, in the γδ TCR, the disulfide bond which is not present in native TCRs is between cysteine residues substituted for a residue of TRGJ located in the following motif:

| | |
|---|---|
| FX₁X₂G | (SEQ ID NO: 2) |

Wherein X₂ denotes the residue to be substituted And Thr 49 of TRDV2.

A specific embodiment of the invention is provided wherein, in the γδ TCR, the disulfide bond which is not present in native TCRs is between cysteine residues substituted for a residue of TRGJ located in the following motif:

| | |
|---|---|
| FX₁X₂G | (SEQ ID NO: 2) |

Wherein X₂ denotes the residue to be substituted

And Glu 49 of TRDV1.

A specific embodiment of the invention is provided wherein, in the γδ TCR, the disulfide bond which is not present in native TCRs is between cysteine residues substituted for a residue of TRGJ located in the following motif:

| | |
|---|---|
| FX₁X₂G | (SEQ ID NO: 2) |

Wherein X₂ denotes the residue to be substituted

And Ser 49 of TRDV3.

A further embodiment is provided by heterodimeric γδTCRs wherein (i) and (ii) are linked by a disulfide bond between cysteine residues present in peptide sequences fused at the C-termini of the TCR.

Another embodiment is provided wherein, in the scTCR forms, a peptidic linker sequence links the C terminus of the first TCR chain to the N terminus of the second TCR chain. Said linker may have the formula —PGGG-(SGGGG)₅-P— (SEQ ID NO: 3) or —PGGG-(SGGGG)₆-P— (SEQ ID NO: 4) wherein P is proline, G is glycine and S is serine.

In a specific embodiment of the invention peptide sequences fused at the C-termini of the TCR easily interact to form a covalent bond between an amino acid in the first peptide sequence and an amino acid in the second peptide linking the TCR γ chain and TCR δ chain together.

Another aspect of the invention is provided wherein the γδ TCR of the invention is soluble.

A further aspect of the invention is provided wherein, the soluble γδ TCR comprises all or part of a TCR γ chain except the transmembrane domain thereof and all or part of a TCR δ chain except the transmembrane domain thereof, wherein each TCR chain each comprise the functional variable domain of a first TCR fused to all or part of the constant domain of a second TCR, the first and second TCRs being from the same species.

An additional aspect is provided wherein a soluble γδ TCR of the invention further comprises a detectable label.

In addition to the non-native disulfide bond referred to above, the γδ TCRs of the invention may include a disulfide bond between residues corresponding to those linked by a disulfide bond in native TCRs.

The soluble γδ TCR of the invention preferably do not contain a sequence corresponding to transmembrane and/or cytoplasmic sequences of native TCRs.

### Multivalent Complexes

One aspect of the invention provides a multivalent complex comprising a plurality of γδ TCRs. One embodiment of this aspect is provided by two or three or four associated soluble γδ TCR associated with one another via a linker radical comprising a polyalkylene glycol polymer or a peptidic sequence. Preferably the complexes are water soluble, so the linker radical should be selected accordingly. Furthermore, it is preferable that the linker radical should be capable of attachment to defined positions on the soluble γδ TCRs, so that the structural diversity of the complexes formed is minimised. One embodiment of the present aspect is provided by a multivalent complex of the invention wherein the polymer chain or peptidic linker sequence extends between amino acid residues of each soluble γδ TCR which are not located in a variable region sequence of the soluble γδ TCR thereof.

Since the complexes of the invention may be for use in medicine, the linker moieties should be chosen with due regard to their pharmaceutical suitability, for example their immunogenicity.

Examples of linker moieties which fulfil the above desirable criteria are known in the art, for example the art of linking antibody fragments.

There are two classes of linker that are preferred for use in the production of multivalent complexes of the present invention. A multivalent complex of the invention in which the soluble γδ TCRs are linked by a polyalkylene glycol chain provides one embodiment of the present aspect.

The first are hydrophilic polymers such as polyalkylene glycols. The most commonly used of this class are based on polyethylene glycol or PEG, the structure of which is shown below.

HOCH₂CH₂—O—(CH₂CH₂O)ₙ—CH₂CH₂OH

Wherein n is greater than two. However, others are based on other suitable, optionally substituted, polyalkylene glycols include polypropylene glycol, and copolymers of ethylene glycol and propylene glycol.

Such polymers may be used to treat or conjugate therapeutic agents, particularly polypeptide or protein therapeutics, to achieve beneficial changes to the PK profile of the therapeutic, for example reduced renal clearance, improved plasma half-life, reduced immunogenicity, and improved solubility. Such improvements in the PK profile of the PEG-therapeutic conjugate are believe to result from the PEG molecule or molecules forming a ‘shell’ around the therapeutic which sterically hinders the reaction with the immune system and reduces proteolytic degradation. (Casey et al, (2000) Tumor Targeting 4 235-244) The size of the hydrophilic polymer used my in particular be selected on the basis of the intended therapeutic use of the TCR complex. Thus for example, where the product is intended to leave the circulation and penetrate tissue, for example for use in the treatment of a tumour, it may be advantageous to use low molecular weight polymers in the order of 5 KDa. There are numerous review papers and books that detail the use of PEG and similar molecules in pharmaceutical formulations. For example, see Harris (1992) Polyethylene Glycol Chemistry—Biotechnical and Biomedical Applications, Plenum, New York, N.Y. or Harris & Zalipsky (1997) Chemistry and Biological Applications of Polyethylene Glycol ACS Books, Washington, D.C.,

The polymer used can have a linear or branched conformation. Branched PEG molecules, or derivatives thereof, can be induced by the addition of branching moieties including glycerol and glycerol oligomers, pentaerythritol, sorbitol and lysine.

Usually, the polymer will have a chemically reactive group or groups in its structure, for example at one or both termini, and/or on branches from the backbone, to enable the polymer to link to target sites in the soluble γδ TCR. This chemically reactive group or groups may be attached directly to the hydrophilic polymer, or there may be a spacer group/moiety between the hydrophilic polymer and the reactive chemistry as shown below:
Reactive chemistry-Hydrophilic polymer-Reactive chemistry
Reactive chemistry-Spacer-Hydrophilic polymer-Spacer-Reactive chemistry

The spacer used in the formation of constructs of the type outlined above may be any organic moiety that is a non-reactive, chemically stable, chain, Such spacers include, by are not limited to the following:

—(CH₂)ₙ— wherein n=2 to 5

—(CH₂)₃NHCO(CH₂)₂

A multivalent complex of the invention in which a divalent alkylene spacer radical is located between the polyalkylene glycol chain and its point of attachment to a soluble γδ TCR of the complex provides a further embodiment of the present aspect.

A multivalent complex of the invention in which the polyalkylene glycol chain comprises at least two polyethylene glycol repeating units provides a further embodiment of the present aspect.

There are a number of commercial suppliers of hydrophilic polymers linked, directly or via a spacer, to reactive chemistries that may be of use in the present invention. These suppliers include Nektar Therapeutics (CA, USA), NOF Corporation (Japan), Sunbio (South Korea) and Enzon Pharmaceuticals (NJ, USA).

Commercially available hydrophilic polymers linked, directly or via a spacer, to reactive chemistries that may be of use in the present invention include, but are not limited to, the following:

| PEG linker | | Catalogue |
|---|---|---|
| Description | Source of PEG | Number |
| Dimer linkers | | |
| 3.4K linear (Maleimide) | Nektar | 2D2DOFO2 |
| 5K forked (Maleimide) | Nektar | 2D2DOHOF |
| 10K linear (with orthopyridyl ds- | Sunbio | |
| linkers in place of Maleimide) | | |
| 20K forked (Maleimide) | Nektar | 2D2DOPOF |
| 20K linear (Maleimide) | NOF Corporation | |
| 40K forked (Maleimide) | Nektar | 2D3XOTOF |
| Higher order multimer linkers | | |
| 15K, 3 arms, Mal₃ (for trimer) | Nektar | OJOONO3 |
| 20K, 4 arms, Mal₄ (for tetramer) | Nektar | OJOOPO4 |
| 40K, 8 arms, Mal₈ (for octamer) | Nektar | OJOOT08 |

A variety of coupling chemistries can be used to couple polymer molecules to protein and peptide therapeutics. The choice of the most appropriate coupling chemistry is largely dependent on the desired coupling site. For example, the following coupling chemistries have been used attached to one or more of the termini of PEG molecules (Source: Nektar Molecular Engineering Catalogue 2003):
N-maleimide
Vinyl sulfone
Benzotriazole carbonate
Succinimidyl proprionate
Succinimidyl butanoate
Thio-ester
Acetaldehydes
Acrylates
Biotin
Primary amines

As stated above non-PEG based polymers also provide suitable linkers for multimerising the soluble γδ TCRs of the present invention. For example, moieties containing maleimide termini linked by aliphatic chains such as BMH and BMOE (Pierce, products Nos. 22330 and 22323) can be used.

Peptidic linkers are the other preferred class of linker radicals. These linkers are comprised of chains of amino acids, and function to produce simple linkers or multimerisation domains onto which soluble γδ TCRs can be attached. The biotin/streptavidin system has previously been used to produce tetramers of soluble TCRs (see WO 99/60119) for in-vitro binding studies. However, stepavidin is a microbially-derived polypeptide and as such not ideally suited to use in a therapeutic.

A multivalent complex of the invention in which the soluble γδ TCRs are linked by a peptidic linker derived from a human multimerisation domain provides a further embodiment of the present aspect.

There are a number of human proteins that contain a multimerisation domain that could be used in the production of such multivalent complexes. For example the tetramerisation domain of p53 which has been utilised to produce tetramers of scFv antibody fragments which exhibited increased serum persistence and significantly reduced off-rate compared to the monomeric scFV fragment. (Willuda et al. (2001) J. Biol. Chem. 276 (17) 14385-14392) Haemoglobin also has a tetramerisation domain that could potentially be used for this kind of application.

A multivalent complex comprising at least two soluble γδ TCRs wherein at least one of said soluble γδ TCRs is a soluble γδ TCR of the invention provides another embodiment of this aspect.

### Uses of the γδ TCRs of the Invention

One aspect the invention provides a method for identifying cells which present γδ TCR ligand complexes, which comprises:
(i) providing a soluble γδ TCR of the invention or a multivalent complex thereof, or cells expressing on the surface thereof at least one γδ TCR of the invention;
(ii) contacting the soluble γδ TCR or multivalent γδ TCR complex thereof, or cells expressing on the surface thereof at least one γδ TCR of the invention with the a diverse population of cells; and
(iii) detecting binding of the soluble γδ TCR or multivalent γδ TCR complex thereof, or cells expressing on the surface thereof at least one γδ TCR of the invention to the cells which present a TCR ligand complex.

Specific embodiments of this aspect are provided wherein the soluble γδ TCR and/or the diverse population of cells are obtained from a subject suffering from a given disease or disorder.

Also provided is a method for delivering a γδ TCR of the invention to a target cell, which method comprises contacting potential target cells with a soluble γδ TCR or multivalent complex, or cells expressing on the surface thereof at least one γδ TCR of the invention under conditions to allow attachment of the soluble γδ TCR or multivalent complex, or cells expressing on the surface thereof at least one γδ TCR of the invention to the target cell, said soluble γδ TCR or multivalent complex, or cells expressing on the surface thereof at least one γδ TCR of the invention being specific for a given γδ TCR ligand. In a specific embodiment of this aspect soluble Vγ9 Vδ2 TCRs or multivalent complexes thereof, or cells of the invention expressing said Vγ9 Vδ2 TCR are used to target cancer cells.

A soluble γδ TCR (or multivalent complex thereof) of the present invention may alternatively or additionally be associated with (e.g. covalently or otherwise linked to) a therapeutic agent which may be, for example, a toxic moiety for use in cell killing, or an immunostimulating agent such as an interleukin or a cytokine. A multivalent soluble γδ TCR complex of the present invention may have enhanced binding capability for a TCR ligand compared to a non-multimeric wild-type or high affinity T cell receptor heterodimer. Thus, the multivalent soluble γδ TCR complexes according to the invention are particularly useful for tracking or targeting cells presenting particular antigens in vitro or in vivo, and are also useful as intermediates for the production of further soluble γδ TCR complexes having such uses. The soluble γδ TCR or multivalent complexes thereof may therefore be provided in a pharmaceutically acceptable formulation for use in vivo.

The invention also provides a method for delivering a therapeutic agent to a target cell, which method comprises contacting potential target cells with a soluble γδ TCR or multivalent complexes thereof in accordance with the invention under conditions to allow attachment of the soluble γδ TCR or multivalent complexes thereof to the target cell, said soluble γδ TCR or multivalent complexes thereof being specific for the TCR ligand and having the therapeutic agent associated therewith.

In particular, the soluble γδ TCR or multivalent complexes thereof of the present invention can be used to deliver therapeutic agents to the location of cells presenting a particular antigen. This would be useful in many situations and, in particular, against tumours. A therapeutic agent could be delivered such that it would exercise its effect locally but not only on the cells has it bound. Thus, one particular strategy envisages anti-tumour molecules linked to a soluble γδ TCR or multivalent complexes thereof specific for tumour antigens. In a specific embodiment of this aspect soluble Vγ9 Vδ2 TCRs or multivalent complexes thereof are used to deliver therapeutic agents to cancer cells.

Many therapeutic agents could be employed for this use, for instance radioactive compounds, enzymes (perforin for example) or chemotherapeutic agents (cis-platin for example). To ensure that toxic effects are exercised in the desired location the toxin could be inside a liposome linked to streptavidin so that the compound is released slowly. This will prevent damaging effects during the transport in the body and ensure that the toxin has maximum effect after binding of the TCR to the relevant antigen presenting cells.

Other suitable therapeutic agents include:
small molecule cytotoxic agents, i.e. compounds with the ability to kill mammalian cells having a molecular weight of less than 700 daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolmide, topotecan, trimetreate glucuronate, auristatin E vincristine and doxorubicin;
peptide cytotoxins, i.e. proteins or fragments thereof with the ability to kill mammalian cells. Examples include ricin, diphtheria toxin, pseudomonas bacterial exotoxin A, DNAase and RNAase;
radio-nuclides, i.e. unstable isotopes of elements which decay with the concurrent emission of one or more of α or β particles, or γ rays. Examples include iodine 131, rhenium 186, indium 111, yttrium 90, bismuth 210 and 213, actinium 225 and astatine 213; chelating agents may be used to facilitate the association of these radio-nuclides to the high affinity TCRs, or multimers thereof;
prodrugs, such as antibody directed enzyme pro-drugs;
immuno-modulatory agents, i.e. moieties which modulate an immune response. Examples include IL-1, IL-1α, IL-2. IL-3, IL-4, IL-5, IL-6, IL-7, IL-8, IL-10, IL-11, IL-12, IL-13, IL-15, IL-21, TGF-β, Lymphotoxin, TNFα, “Anti-T cell antibodies” such as Anti-CD3 antibody or Anti-CD4 antibody or Anti-CD8 antibody or Anti-CD25 antibody or Anti-CD28 antibody, Anti-CD45RA antibody, Anti-CD45RB antibody, Anti-CD44 antibody, Anti-Thy 1.2 antibody, Antilymphocyte globulin, Anti-αβTCR antibody, Anti-Vβ8 antibody, Anti-CD16 antibody, CTLA-4-Ig, Anti-B7.2 antibody, Anti-CD40L antibody, Anti-ICAM-1 antibody, ICAM-1, Anti-Mac antibody, Anti-LFA-1 antibody, Anti-IFN-γ antibody IFN-γ, IFN-γR/IgG1 fusions, Anti-IL-2R antibodies, IL-2R antibody, IL-2 Diptheria-toxin protein, Anti-IL-12 antibody, IL-12 Antagonist (p40), Anti-IL-1 antibody, IL-1 Antagonist, Glutamic acid decarboxylase (GAD), Anti-GAD antibody, Viral proteins and peptides, Bacterial proteins or peptides, A-Glactoslyceramide, Calcitonin, Nicotinamide, Anti-oxidants (Vitamin E, Probucol analog, Probucol+deflazacoert or Aminoguanidine), Anti-Inflammatory agents (Pentoxifylline or Rolipram), immunomodulators (Linomide, Ling-zhi-8, D-Glucan, Multi-functional protein 14, Ciamexon, Cholera toxin B, Vanadate or Vitamin D3 analogue, small molecule CD80 inhibitors, Androgens, IGF-1, Immunomanipulation (Natural antibodies), Lupus idiotype, Lipopolysaccaride), Sulfatide, Bee venom, Kampo formulation, Silica, Ganglioside, Antiasialo GM-1 antibody, Hyaluronidase, Concanavalin A, Anti-Class I MHC antibody, or Anti-Class II MHC antibody, Cyclosporin, FK-506, Azathioprine, Rapamycin or Deoxyspergualin, or a functional variant or fragment of any of the foregoing

In particular, the soluble γδ TCR or multivalent complex of the present invention can be used to deliver superantigens to the location of cells presenting a particular antigen. This is useful in many situations, for example, against tumours or sites of infectious disease. A superantigen can be delivered such that it exercises its effect locally but not only on the cell to which it binds.

Thus, one particular strategy uses soluble γδ TCRs or multivalent complexes according to the invention specific for tumour antigens. For cancer treatment, the localisation in the vicinity of tumours or metastasis enhances the effect of the therapeutic agent. Alternatively, the soluble γδ TCRs or multivalent complexes of the present invention can be used to deliver therapeutic agents to the location of cells presenting a particular antigen related to an infectious disease.

Further embodiments of the invention are provided by a pharmaceutical composition comprising a soluble γδ TCR or multivalent complex of the invention, or cells expressing on the surface thereof at least one γδ TCR of the invention together with a pharmaceutically acceptable carrier.

The invention also provides a method of treatment of cancer comprising administering to a subject suffering such cancer an effective amount of a soluble γδ TCR or multivalent complex of the invention or cells expressing on the surface thereof at least one γδ TCR of the invention. In a related embodiment, the invention provides for the use of a soluble γδ TCR or multivalent complex of the invention, or cells expressing on the surface thereof at least one γδ TCR of the invention in the preparation of a composition for the treatment of cancer. Vγ9 Vδ2 TCRs are preferred γδ TCRs for use such cancer-related methods and compositions.

The invention also provides a method of treatment of autoimmune disease, organ rejection or GVHD comprising administering to a subject suffering such autoimmune disease, organ rejection or GVHD an effective amount of a soluble γδ TCR or multivalent complex of the invention, or cells expressing on the surface thereof at least one γδ TCR of the invention. In a related embodiment, the invention provides for the use of a soluble γδ TCR or multivalent complex of the invention, or cells expressing on the surface thereof at least one γδ TCR of the invention in the preparation of a composition for the treatment of autoimmune disease, organ rejection or GVHD.

The invention also provides a method of treatment of infectious disease comprising administering to a subject suffering such an infectious disease an effective amount of a soluble γδ TCR or multivalent complex of the invention, or cells expressing on the surface thereof at least one γδ TCR of the invention. In a related embodiment the invention provides for the use of a soluble γδ TCR or multivalent complex of the invention, or cells expressing on the surface thereof at least one γδ TCR of the invention in the preparation of a composition for the treatment of infectious disease.

Cancers for treatment by the compositions and methods of the present invention include, but are not limited to; leukaemia, head and neck, lung, breast, colon, cervical, liver, pancreatic, ovarian, prostate, colon, liver, bladder, oesophageal, stomach, melanoma and testicular.

Infectious diseases for treatment by the compositions and methods of the present invention are those caused by intracellular infectious organisms. The term “intracellular infectious organisms” as used herein is understood to encompass any organisms capable of entering a human cell. Such organisms may cause disease directly, or directly leading to altered cell function. These organisms can be any of the following:

Bacteria, Fungi, Viruses, Protozoa and Mycobacteria.

Examples of these diseases and the intracellular infectious organisms which cause them include, but are not limited to listeriosis caused by Listeria monocytogenes, bubonic plague caused by the Yersintia pestis bacteria and T-cell leukemia cause by the HTLV-1 virus.

Auto-immune diseases which may benefit the methods of the following invention include:

Acute disseminated encephalomyelitis
Adrenal insufficiency
Allergic angiitis and granulomatosis

### Amylodosis

Ankylosing spondylitis

### Asthma

Autoimmune Addison's disease
Autoimmune alopecia
Autoimmune chronic active hepatitis
Autoimmune haemolytic anaemia

### Autoimmune Neutrogena

Autoimmune thrombocytopenic purpura
Behçet's disease
Cerebellar degeneration
Chronic active hepatitis
Chronic inflammatory demyelinating polyradiculoneuropathy
Chronic neuropathy with monoclonal gammopathy
Classic polyarteritis nodosa
Congenital adrenal hyperplasia

### Cryopathies

Dermatitis herpetiformis

### Diabetes

Eaton-Lambert myasthenic syndrome

### Encephalomyelitis

Epidermolysis bullosa acquisita
Erythema nodosa
Gluten-sensitive enteropathy
Goodpasture's syndrome
Guillain-Barre syndrome
Hashimoto's thyroiditis

### Hyperthyroidism

Idiopathic hemachromatosis
Idiopathic membranous glomerulonephritis
Isolated vasculitis of the central nervous system
Kawasald's disease
Minimal change renal disease
Miscellaneous vasculitides
Mixed connective tissue disease
Multifocal motor neuropathy with conduction block
Multiple sclerosis
Myasthenia gravis
Opsoclonus-myoclonus syndrome

### Pemphigoid

### Pemphigus

pernicious anaemia

### Polymyositis/dermatomyositis

Post-infective arthritides
Primary biliary sclerosis

### Psoriasis

Reactive arthritides
Reiter's disease

### Retinopathy

Rheumatoid arthritis
Sclerosing cholangitis
Sjögren's syndrome
Stiff-man syndrome
Subacute thyroiditis
Systemic lupus erythematosis
Systemic necrotizing vasculitides
Systemic sclerosis (scleroderma)
Takayasu's arteritis
Temporal arteritis
Thromboangiitis obliterans
Type I and type II autoimmune polyglandular syndrome
Ulcerative colitis

### Uveitis

Wegener's granulomatosis

Therapeutic soluble γδ TCRs or multivalent complexes in accordance with the invention, or cells expressing on the surface thereof at least one γδ TCR of the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example parenteral, transdermal or via inhalation, preferably a parenteral (including subcutaneous, intramuscular, or, most preferably intravenous) route. Such compositions may be prepared by any method known in the art of pharmacy, for example by admixing the active ingredient with the carrier(s) or excipient(s) under sterile conditions.

Dosages of the substances of the present invention can vary between wide limits, depending upon the disease or disorder to be treated, the age and condition of the individual to be treated, etc. and a physician will ultimately determine appropriate dosages to be used.

Gene cloning techniques may be used to provide a soluble γδ TCR of the invention, preferably in substantially pure form. These techniques are disclosed, for example, in J. Sambrook et al Molecular Cloning 2nd Edition, Cold Spring Harbor Laboratory Press (1989). Thus, in a further aspect, the present invention provides a nucleic acid molecule comprising a sequence encoding a chain of the soluble TCR of the present invention, or a sequence complementary thereto. Such nucleic acid sequences may be obtained by making appropriate mutations (by insertion, deletion or substitution) to TCR-encoding nucleic acid isolated from T-cell clones or by de-novo synthesis of published γδ TCR DNA sequences.

The nucleic acid molecule may be in isolated or recombinant form. It may be incorporated into a vector and the vector may be incorporated into a host cell. Such vectors and suitable hosts form yet further aspects of the present invention.

The invention also provides a method for obtaining a soluble γ TCR or δ TCR chain, which method comprises incubating such a host cell under conditions causing expression of the TCR chain and then purifying the polypeptide.

The soluble γδ TCRs of the present invention may obtained by expression in a bacterium such as E. coli as inclusion bodies, and subsequent refolding in vitro.

Refolding of the soluble γδ TCR chains may take place in vitro under suitable refolding conditions. In a particular embodiment, a TCR with correct conformation is achieved by refolding solubilised TCR chains in a refolding buffer comprising a solubilising agent, for example guanidine. Advantageously, the guanidine may be present at a concentration of at least 0.1M or at least 1M or at least 2.5M, or about 6M. An alternative solubilising agent which may be used is urea, at a concentration of between 0.1M and 8M, preferably at least 1M or at least 2.5M. Prior to refolding, a reducing agent is preferably employed to ensure complete reduction of cysteine residues.

As is known to those skilled in the art the refolding methods utilised be varied in order to optimise the yield of refolded protein obtained. For example, further denaturing agents such as DTT and guanidine may be used as necessary. Alternatively or additionally, different denaturants and reducing agents may be used prior to the refolding step (e.g. urea, β-mercaptoethanol). Alternatively or additionally, redox couples may be used during refolding, such as a cystamine/cysteamine redox couple, DTT or β-mercaptoethanol/atmospheric oxygen, and cysteine in reduced and oxidised forms.

Folding efficiency may also be increased by the addition of certain other protein components, for example chaperone proteins, to the refolding mixture. Improved refolding has been achieved by passing protein through columns with immobilised mini-chaperones (Altamirano, et al. (1999). Nature Biotechnology 17: 187-191; Altamirano, et al. (1997). Proc Natl Acad Sci USA 94 (8): 3576-8).

Alternatively, soluble TCR of the present invention may obtained by expression in a eukaryotic cell system, such as insect cells.

Purification of the soluble TCR may be achieved by many different means. Alternative modes of ion exchange may be employed or other modes of protein purification may be used such as gel filtration chromatography or affinity chromatography.

### Additional Aspects

A soluble γδ TCR or multivalent complex of the present invention may be provided in substantially pure form, or as a purified or isolated preparation. For example, it may be provided in a form which is substantially free of other proteins.

Preferred features of each aspect of the invention are as for each of the other aspects mutatis mutanidis. The prior art documents mentioned herein are incorporated to the fullest extent permitted by law.

### EXAMPLES

The invention is further described in the following examples, which do not limit the scope of the invention in any way.

Reference is made in the following to the accompanying drawings in which:

FIG. 1 provides a comparison of the structures of a soluble disulfide-linked αβ TCR and a soluble γδ TCR.

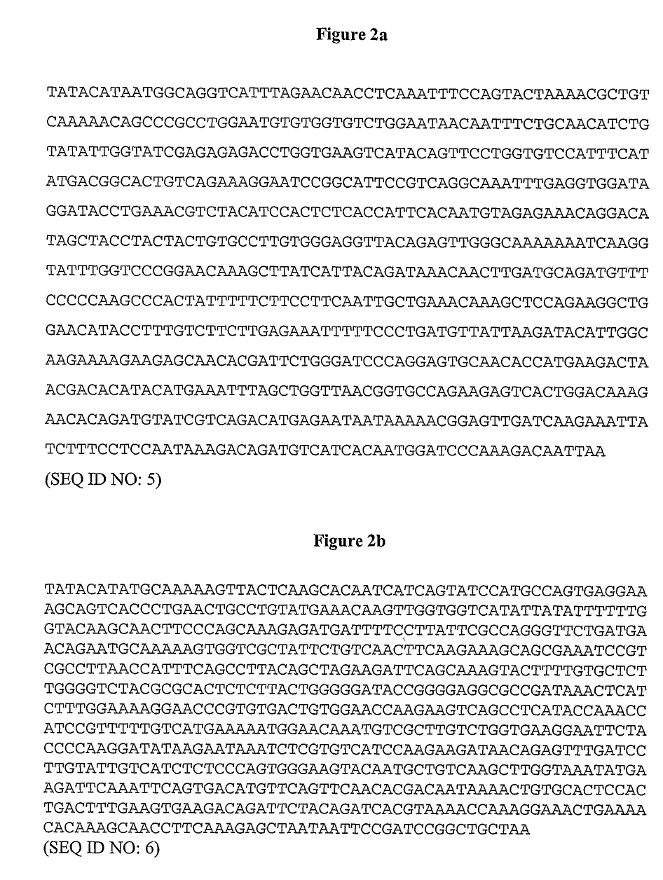

FIGS. 2a and 2b detail the DNA sequences of soluble TCR γ and δ chains respectively of the soluble W γδ TCR mutated incorporate an introduced disulfide interchain bond between TRGC1 Exon 1 residue 60 and TRDC Exon 1 Residue 47.

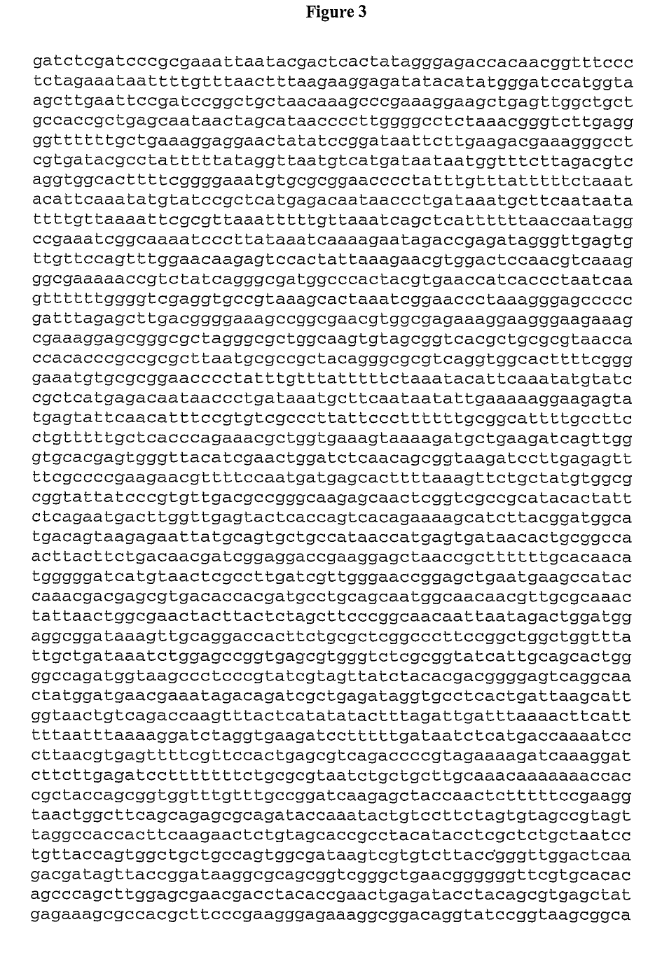

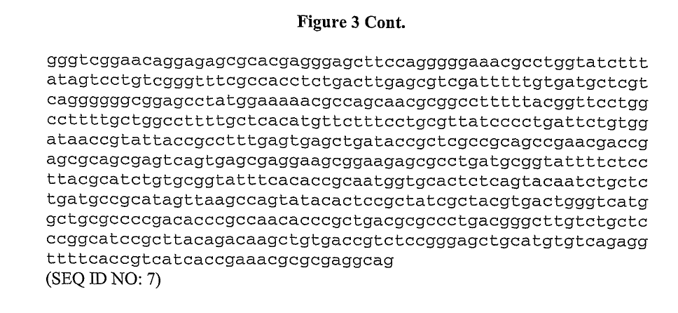

FIG. 3 details the DNA sequence of the pGMT7 plasmid

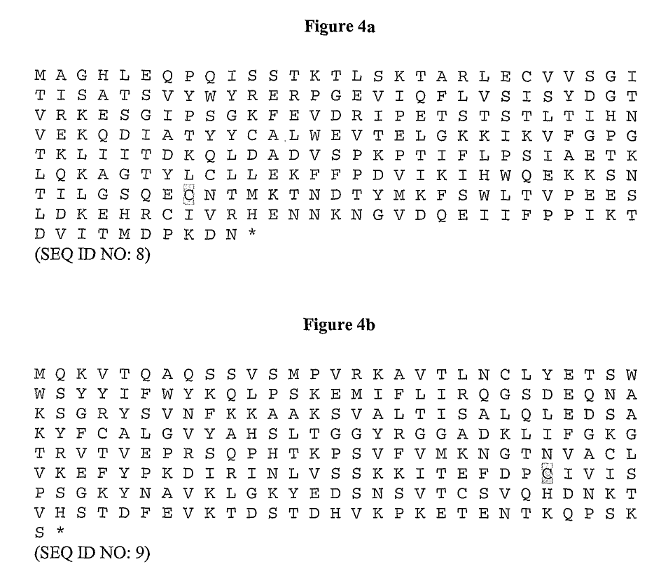

FIGS. 4a and 4b detail the amino acid sequences of the TCR γ and δ chains encoded by the DNA sequences of FIGS. 2a and 2b respectively. The introduced cysteine residues are indicated by shading.

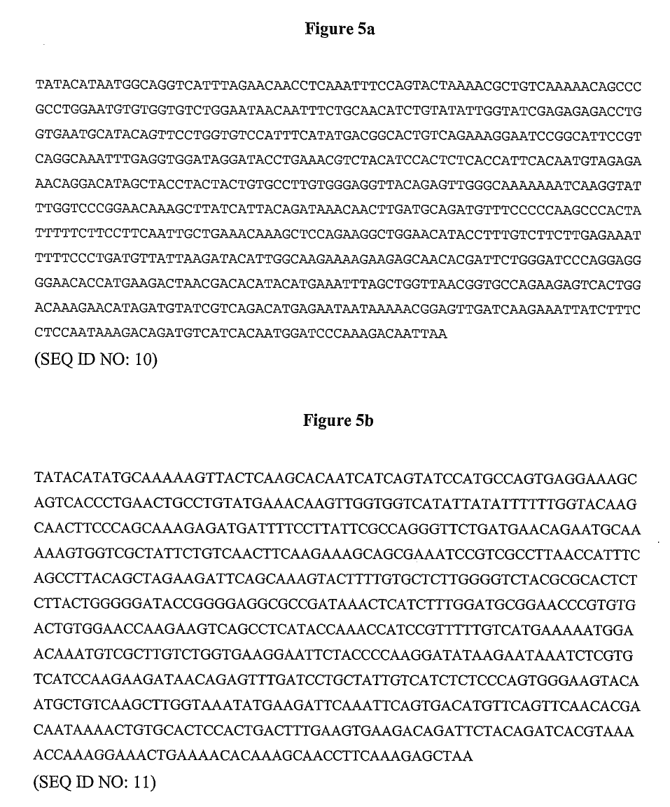

FIGS. 5a and 5b detail the DNA sequences of soluble TCR γ and δ chains respectively of the soluble W γδ TCR mutated to incorporate an introduced disulfide interchain bond between TRGV9 Residue 49 and a residue of TRDJ1 located in a motif FGXG (SEQ ID NO: 1) wherein X denotes the residue to be substituted.

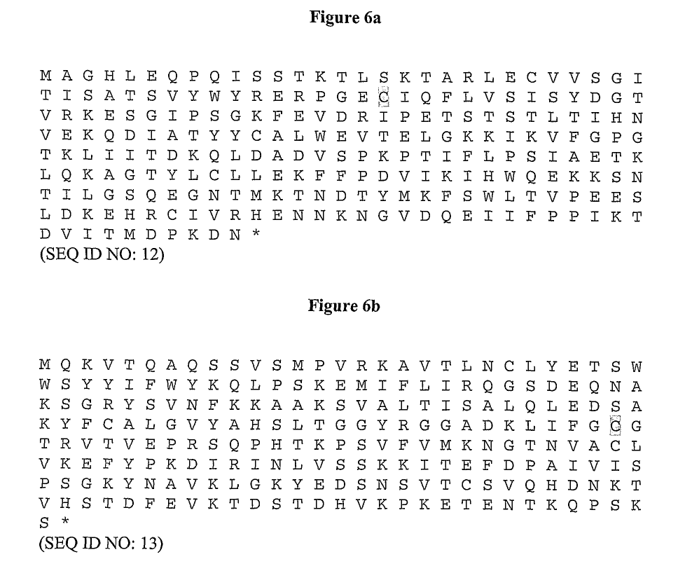

FIGS. 6a and 6b detail the amino acid sequences encoded by the DNA sequences of FIGS. 5a and 5b respectively. The introduced cysteine residues are indicated by shading.

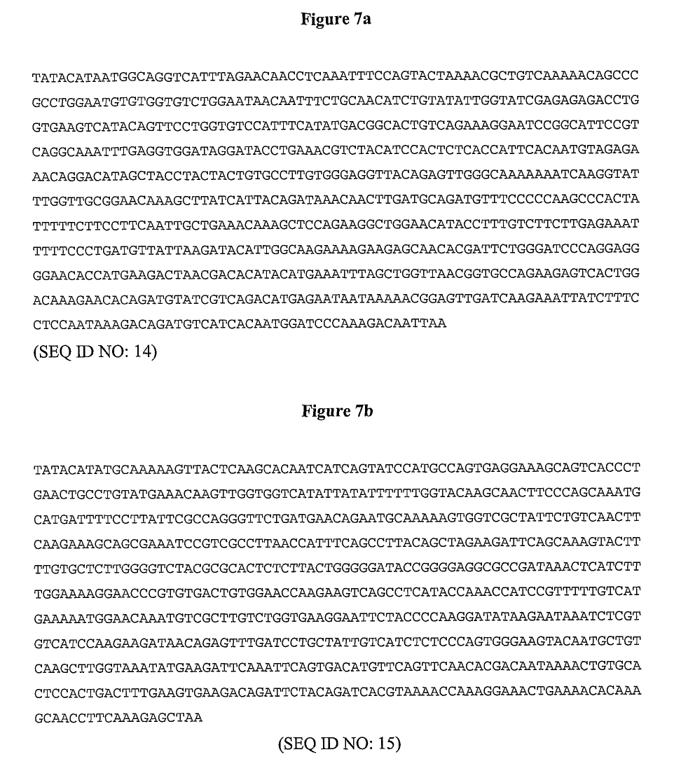

FIGS. 7a and 7b detail the DNA sequences of soluble TCR γ and δ chains respectively of the soluble W γδ TCR mutated to incorporate an introduced disulfide interchain bond between TRGJP Residue 13 and TRDV1 Residue 49.

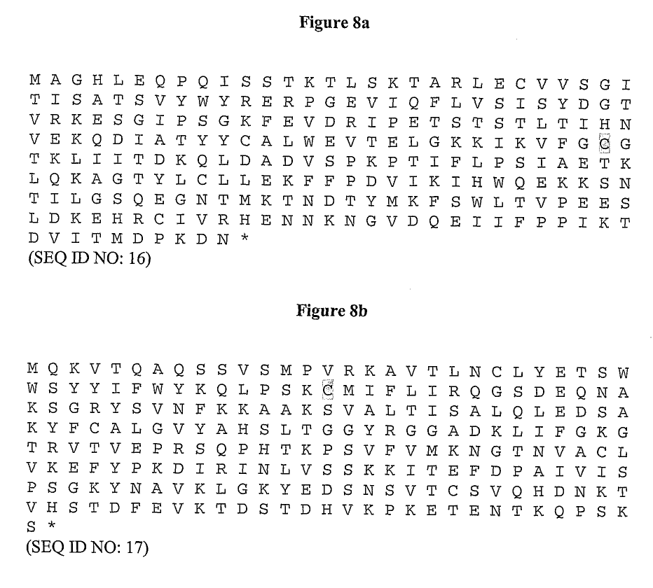

FIGS. 8a and 8b detail the amino acid sequences encoded by the DNA sequences of 7a and 7b respectively. The introduced cysteine residues are indicated by shading.

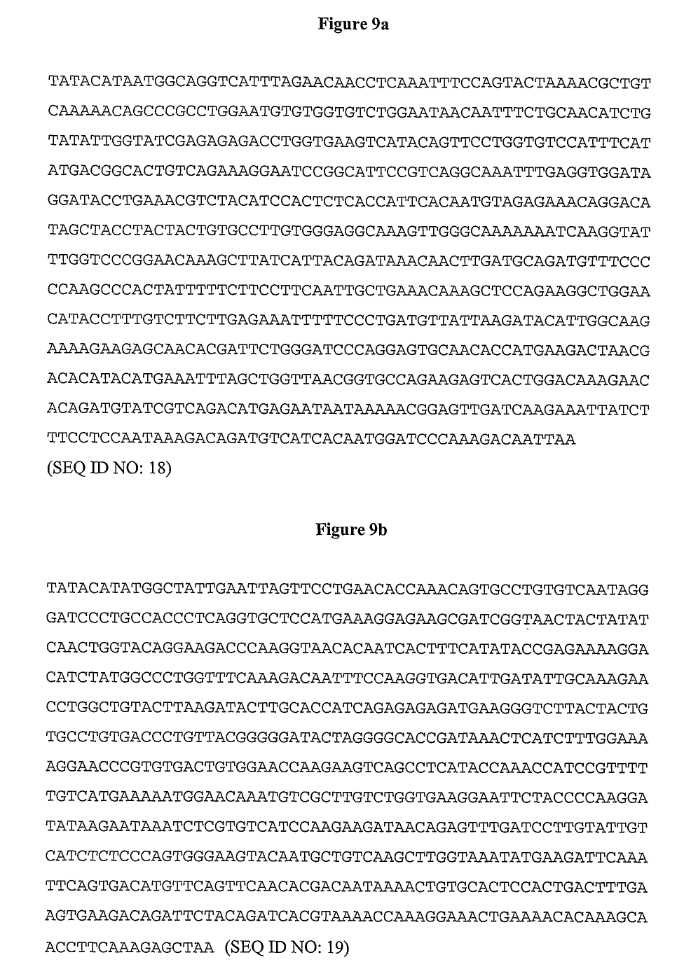

FIGS. 9a and 9b detail the DNA sequences of soluble TCR γ and δ chains respectively of the soluble B γδ TCR mutated to incorporate an introduced disulfide interchain bond between TRGC1 Exon 1 residue 60 and TRDC Exon 1 Residue 47.

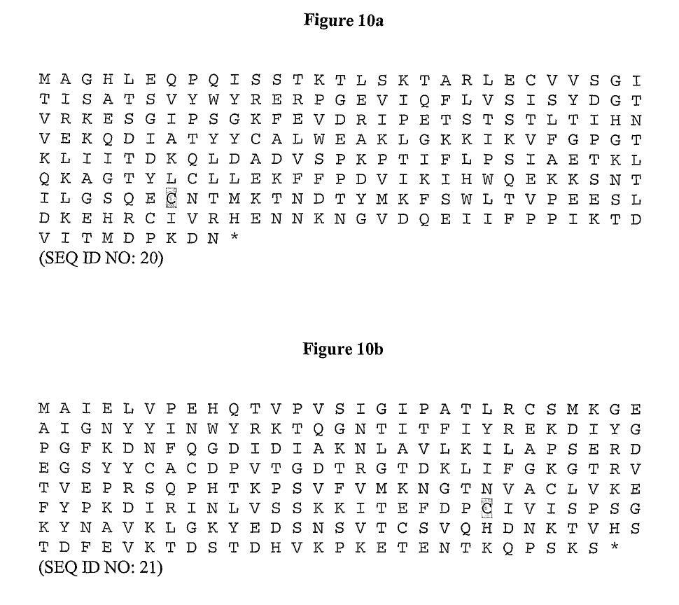

FIGS. 10a and 10b detail the amino acid sequences encoded by the DNA sequences of 9a and 9b respectively. The introduced cysteine residues are indicated by shading.

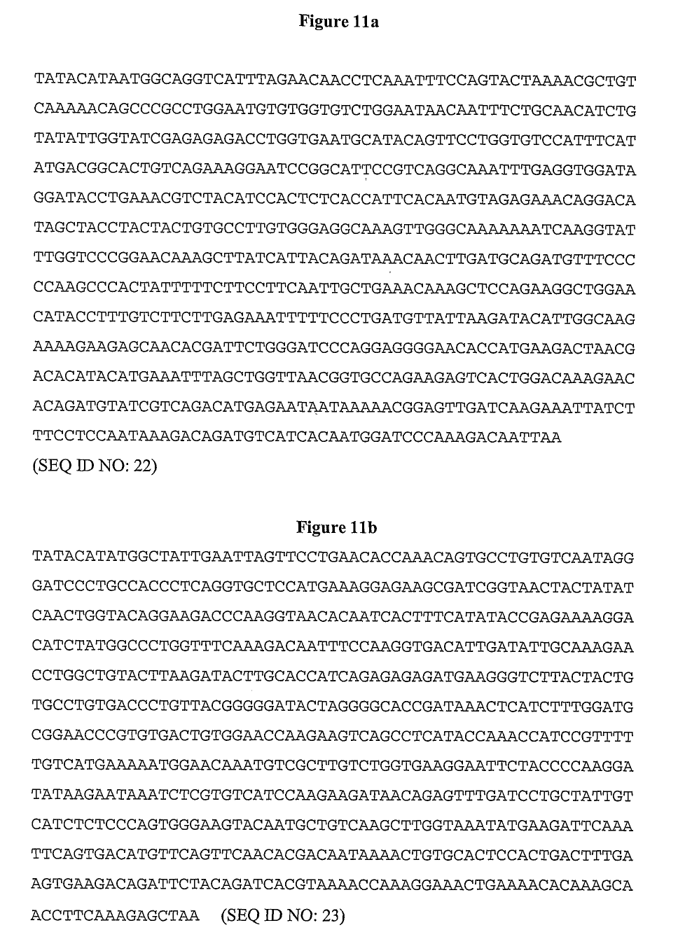

FIGS. 11a and 11b detail the DNA sequences soluble TCR γ and δ chains respectively of the soluble Bγδ TCR mutated to incorporate an introduced disulfide interchain bond between TRGV9 Residue 49 and a residue of TRDJ1 located in a motif FGXG (SEQ ID NO: 1) wherein X denotes the residue to be substituted.

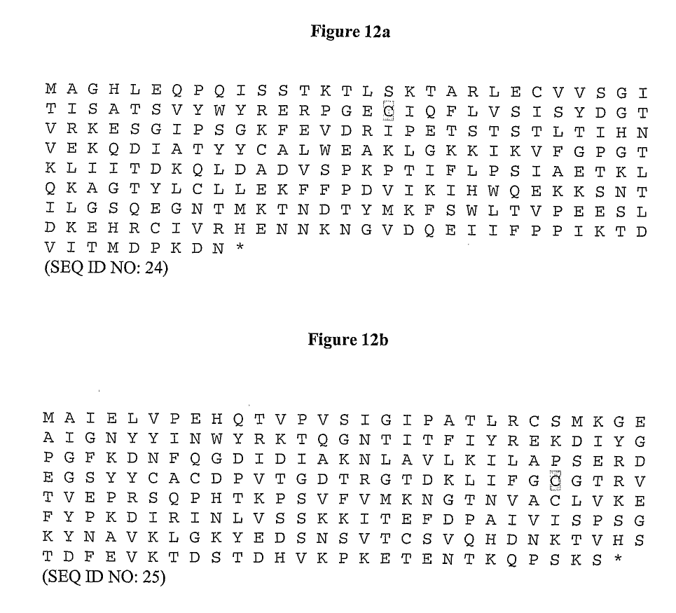

FIGS. 12a and 12b detail the amino acid sequences encoded by the DNA sequences of 11a and 11b respectively. The introduced cysteine residues are indicated by shading.

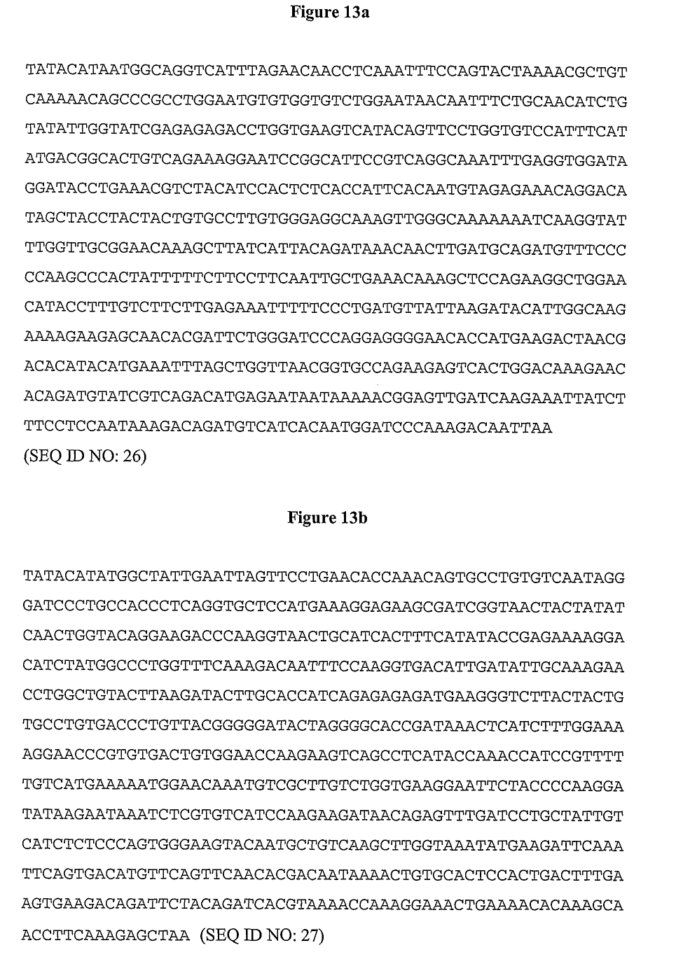

FIGS. 13a and 13b detail DNA sequences of the soluble TCR γ and δ chains respectively of the soluble Bγδ TCR mutated to incorporate an introduced disulfide interchain bond between TRGJP Residue 13 and TRDV2 Residue 49.

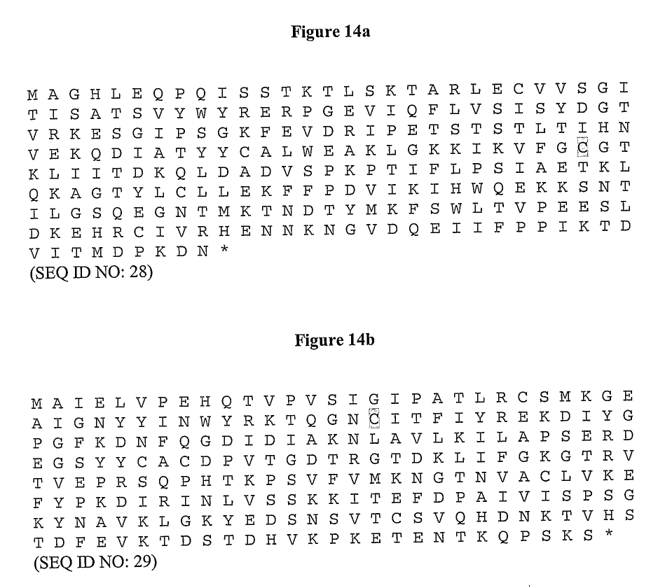

FIGS. 14a and 14b detail the amino acid sequences encoded by the DNA sequences of 13a and 13b respectively. The introduced cysteine residues are indicated by shading.

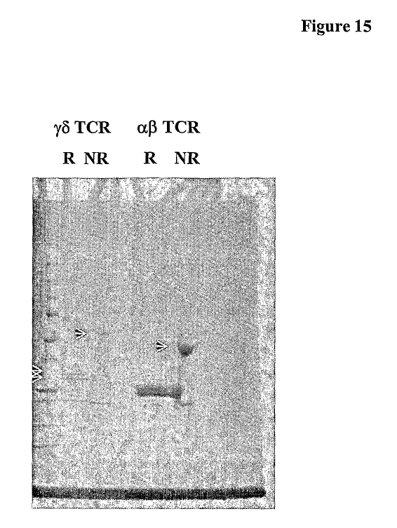

FIG. 15 is an SDS-PAGE gel which compares the bands seen for the γδ TCR comprising the amino acid sequences detailed in FIGS. 4a and 4b with those seen for an αβ TCR. The gel was run on reduced “R” and non-reduced “NR” TCR samples.

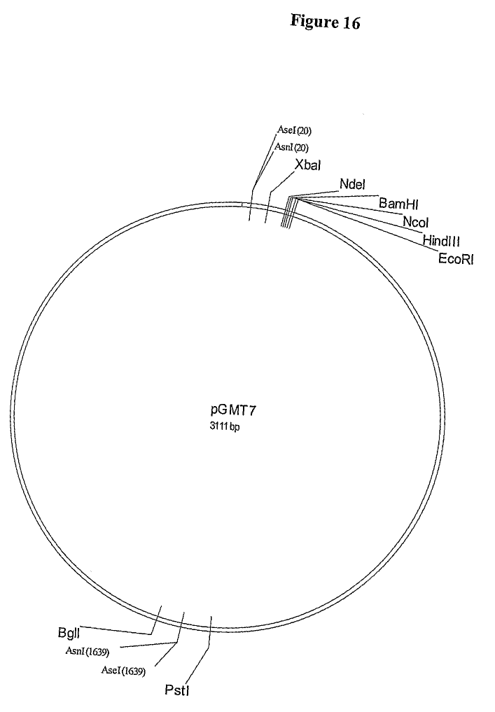

FIG. 16 provides the plasmid map of the pGMT7 vector.

### Example I

### Production of DNA Encoding a Soluble γ, TCR Containing an Introduced Disulfide Interchain Bond

The γδ TCRs which were used as the basis for the modified γδ TCRs in the following examples have been previously described. (Green et al., (2004) Clin Exp Immunol (2004) 136 472-482) The sequences encoding the TCR chains of these γδ TCRs as described in this paper have been placed in the EMBL Nucleotide Sequence Database with the following accession numbers:

| | |
|---|---|
| “B γδ TCR” | γ chain: Accession No. - AJ583012 |
| | δ chain: Accession No. - AJ583013 |
| “W γδ TCR” | γ chain: Accession No. - AJ583014 |
| | δ chain: Accession No. - AJ583915 |

Synthetic genes comprising the DNA sequence encoding the soluble TCR γ and δ chains detailed in FIGS. 2a and 2b respectively were synthesised. This W γδ TCR contains an introduced disulfide interchain bond between TRGC1 Exon 1 residue 60 and TRDC Exon 1 Residue 47.

There are a number of companies that provide a suitable DNA service, such as Geneart (Germany)

The above synthetic genes encoding the soluble TCR γ and δ chains were then separately sub-cloned into pGMT7 plasmids. FIG. 3 details the DNA sequence of the pGMT7 plasmid and FIG. 16 provides the plasmid map of this vector.

FIGS. 4a and 4b detail the amino acid sequences of the TCR γ and δ chains encoded by the DNA sequences of FIGS. 2a and 2b respectively.

The same techniques can be used to produce DNA encoding other soluble γδ TCRs such as the following:

FIGS. 5a and 5b detail the DNA sequences of soluble TCR γ and δ chains respectively of the soluble W γδ TCR mutated to incorporate an introduced disulfide interchain bond between TRGV9 Residue 49 and a residue of TRDJ1 located in a motif FGXG (SEQ ID NO: 1) wherein X denotes the residue to be substituted.

FIGS. 6a and 6b detail the amino acid sequences encoded by the DNA sequences of FIGS. 5a and 5b respectively.

FIGS. 7a and 7b detail the DNA sequences of soluble TCR γ and δ chains respectively of the soluble W γδ TCR mutated to incorporate an introduced disulfide interchain bond between TRGJP Residue 13 and TRDV1 Residue 49.

FIGS. 8a and 8b detail the amino acid sequences encoded by the DNA sequences of 7a and 7b respectively.

FIGS. 9a and 9b detail the DNA sequences of soluble TCR γ and δ chains respectively of the soluble Bγδ TCR mutated to incorporate an introduced disulfide interchain bond between TRGC1 Exon 1 residue 60 and TRDC Exon 1 Residue 47.

FIGS. 10a and 10b detail the amino acid sequences encoded by the DNA sequences of 9a and 9b respectively.

FIGS. 11a and 11b detail the DNA sequences soluble TCR γ and δ chains respectively of the soluble Bγδ TCR mutated to incorporate an introduced disulfide interchain bond between TRGV9 Residue 49 and a residue of TRDJ1 located in a motif FGXG (SEQ ID NO: 1) wherein X denotes the residue to be substituted.

FIGS. 12a and 12b detail the amino acid sequences encoded by the DNA sequences of 11a and 11b respectively.

FIGS. 13a and 13b detail DNA sequences of the soluble TCR γ and δ chains respectively of the soluble Bγδ TCR mutated to incorporate an introduced disulfide interchain bond between TRGJP Residue 13 and TRDV2 Residue 49.

FIGS. 14a and 14b detail the amino acid sequences encoded by the DNA sequences of 13a and 13b respectively.

FIG. 15 is an SDS-PAGE gel which compares the bands seen for the γδ TCR comprising the amino acid sequences detailed in FIGS. 4a and 4b with those seen for an αβ TCR. The gel was run on reduced “R” and non-reduced “NR” TCR samples.

### Example 2

### Expression, Refolding and Purification of Soluble γδ TCRs

pGMT7 (FIG. 3 details the DNA sequence of the pGMT7 plasmid and FIG. 16 provides the plasmid map of this vector) expression plasmids containing the soluble TCR γ and δ chains encoded by FIGS. 1a and 1b respectively were transformed separately into E. coli strain BL21pLysS, and single ampicillin-resistant colonies were grown at 37° C. in TYP (ampicillin 100 μg/ml) medium to OD₆₀₀ of 0.4 before inducing protein expression with 0.5 mM IPTG. Cells were harvested three hours post-induction by centrifugation for 30 minutes at 4100 rpm in a Beckman J-6B. Cell pellets were re-suspended in a buffer containing 50 mM Tris-HCl, 25% (w/v) sucrose, 1 mM NaEDTA, 0.1% (w/v) NaAzide, 10 mM DTT, pH 8.0. After an overnight freeze-thaw step, re-suspended cells were sonicated in 1 minute bursts for a total of around 10 minutes in a Milsonix XL2020 sonicator using a standard 12 mm diameter probe. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl, 0.5% Triton-X100, 200 mM NaCl, 10 mM NaEDTA, 0.1% (w/v)

NaAzide, 2 mM DTT, pH 8.0) before being pelleted by centrifugation for 15 minutes at 13000 rpm in a Beckman J2-21. Detergent and salt was then removed by a similar wash in the following buffer: 50 mM Tris-HCl, 1 mM NaEDTA, 0.1% (w/v) NaAzide, 2 mM DTT, pH 8.0. Finally, the inclusion bodies were divided into 30 mg aliquots and frozen at −70° C. Inclusion body protein yield was quantitated by solubilising with 6M guanidine-HCl and measurement with a Bradford dye-binding assay (PerBio).

Denaturation of the soluble TCR γ and β chains; 30 mg of the solubilised TCR γ chain inclusion body and 30 mg of the solubilised TCR δ chain inclusion body were thawed from frozen stocks. The inclusion bodies were diluted to a final concentration of 5 mg/ml in 6M guanidine solution, and DTT (2M stock) was added to a final concentration of 10 mM. The mixture was incubated at 37° C. for 30 min.

Refolding of the soluble TCR γ and δ chains: 250 ml refolding buffer was stirred vigorously at 5° C.±3° C. The redox couple (2-mercaptoethylamine and cystamine (to final concentrations of 6.6 mM and 3.7 mM, respectively) were added approximately 5 minutes before addition of the denatured TCR γ and δ chains. The soluble γδ TCR was then allowed to refold for approximately 1 hour±15 minutes with stirring at 5° C.±3° C.

Dialysis of the refolded soluble γδ TCR: The refolded soluble γδ TCR was dialysed in Spectrapor 1 membrane (Spectrum; Product No. 132670) against 10 L 10 mM Tris pH 8.1 at 5° C.±3° C. for 18-20 hours. After this time, the dialysis buffer was changed to fresh 10 mM Tris pH 8.1 (10 L) and dialysis was continued at 5° C.±3° C. for another 20-22 hours.

FIG. 15 is an SDS-PAGE gel which compares the bands seen for the refolded γδ TCR comprising the amino acid sequences detailed in FIGS. 4a and 4b with those seen for an at TCR. The gel was run on reduced “R” and non-reduced “NR” TCR samples.

### Example 3

### In-Vitro Cellular Assay for Determining Cells which Present a γδ TCR Ligand

Peripheral blood mononuclear cells (PBMC) are isolated from venous blood samples using a Lymphoprep. The PBMCs are washed and used immediately. Freshly isolated PBMCs are washed twice in 10% autologous human serum/RPMI (Gibco BRL). Finally, the cells are re-suspended in RPMI medium.

5×10⁶ of these PBMCs in RPMI medium are then added to magnetic beads (Dynal Biotech, Norway) which have been coated with (biotinylated) soluble γδ TCRs following the manufacturers instructions. The culture is then incubated for 30 minutes at 37° C., 5% CO₂. During this incubation period cells that present a γδ TCR ligand will adhere to the beads. These adhered cells are then magnetically separated from the rest of the culture.

The above method provides a convenient means by which the proportion of cells from diseased and non-diseased subjects which present a ligand recognized by a given γδ TCR can be quantified. Furthermore, the cell populations isolated can be used to identify the particular γδ TCR ligand presented.

1. A γδ T cell receptor (TCR), which comprises (i) all or part of a TCR γ chain, and (ii) all or part of a TCR δ chain, wherein (i) and (ii) are linked by a disulfide bond which is not present in native γδ TCRs.

2. A TCR as claimed in claim 1, wherein the said disulfide bond is
(i) between:
cysteine residues substituted for residue 60 of exon 1 of TRGC1 or TRGC2(2×) or TRGC2(3×) and residue 47 of exon 1 of TRDC; or
cysteine residues substituted for residue 49 of TRGV and a residue of TRDJ located in a motif FGXG (SEQ ID NO: 1) wherein X denotes the residue to be substituted; or
cysteine residues substituted for a residue of TRGJ located in the following motif:
FX₁X₂G (SEQ ID NO: 2), wherein X₂ denotes the residue to be substituted, and residue 49 of TRDV; or
(ii), only in the case of a heterodimeric γδ TCR, between cysteine residues present in peptide sequences fused at the C-termini of the TCR.

3. A TCR as claimed in claim 1 wherein said TCR is a heterodimeric TCR (dTCR).

4. A TCR as claimed in claim 1 which is a single-chain TCR (scTCR).

5. A TCR as claimed in claim 1 wherein the said disulfide bond is between cysteine residues substituted for TRGC1 Exon 1 residue 60 and TRDC Exon 1 Residue 47.

6. A TCR as claimed in claim 1 which is soluble and lacks any TCR transmembrane domain.

7. A soluble γδ TCR as claimed in claim 6, wherein one or both of the TCR chains are derivatised with, or fused to, a moiety at its C or N terminus.

8. A soluble TCR as claimed in claim 6 comprising a detectable label.

9. A soluble TCR as claimed in claim 6 associated with a therapeutic agent.

10. A multivalent TCR complex comprising a plurality of soluble TCRs as claimed in claim 1.

11. A cell expressing on the surface thereof at least one TCR as claimed in claim 1.

12. A method for identifying cells which present TCR-ligand complexes, which comprises:
(i) providing a TCR as claimed in claim 1 or a multivalent complex thereof or cells expressing on the surface thereof as least one TCR as claimed in claim 1;
(ii) contacting the TCR, the multivalent complex thereof, or the cells with a diverse population of cells; and
(iii) detecting binding of the TCR, the multivalent complex thereof, or the cells to cells which present a TCR ligand complex.

13. A pharmaceutical formulation comprising a TCR as claimed in claim 1, a multivalent TCR complex thereof, or cells expressing on the surface thereof at least one TCR as claimed in claim 1 together with a pharmaceutically acceptable carrier.

14. A method of treatment of cancer comprising administering to a subject suffering such cancer an effective amount of a soluble TCR which lacks any TCR transmembrane domain and which is associated with a therapeutic agent, a multivalent complex thereof or cells expressing on the surface thereof at least one TCR as claimed in claim 1.

15. A method as claimed in claim 14, wherein the TCR is a Vγ9 Vδ2 TCR.

16. (canceled)

17. (canceled)

18. A nucleic acid molecule comprising a sequence encoding a polypeptide selected from (i) and (ii) of a TCR as claimed in claim 1 or a sequence complementary thereto.

19. A vector comprising a nucleic acid molecule as claimed in claim 18.

20. A host cell comprising a vector as claimed in claim 21.

21. A method for obtaining a polypeptide which method comprises incubating a host cell as claimed in claim 20 under conditions causing expression of the peptide and then purifying the polypeptide.

22. A method as claimed in claim 21, further comprising mixing (i) and (ii) under suitable refolding conditions.


SEQUENCE LISTING (29 sequences)

SEQ ID NO: 1 - protein, 4 aa, Artificial sequence
  TRDJ domain motif
FGXG

SEQ ID NO: 2 - protein, 4 aa, Artificial sequence
  TRGJ Motif
FXXG

SEQ ID NO: 3 - protein, 30 aa, Artificial sequence
  Short scTCR linker
PGGGSGGGGSGGGGSGGGGSGGGGSGGGGP

SEQ ID NO: 4 - protein, 35 aa, Artificial sequence
  Long scTCR Linker
PGGGSGGGGSGGGGSGGGGSGGGGSGGGGSGGGGP

SEQ ID NO: 5 - DNA, 737 bp, Artificial sequence
  DNA encoding gamma chain of the soluble DiS linked W gamma-delta TCR
tatacataatggcaggtcatttagaacaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggttacagagttgggcaaaaaaatcaaggtatttggtcccggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggagtgcaacaccatgaagactaacgacacatacatgaaatttagctggttaacggtgccagaagagtcactggacaaagaacacagatgtatcgtcagacatgagaataataaaaacggagttgatcaagaaattatctttcctccaataaagacagatgtcatcacaatggatcccaaagacaattaa

SEQ ID NO: 6 - DNA, 730 bp, Artificial sequence
  DNA encoding delta chain of the soluble DiS linked W gamma-delta TCR
tatacatatgcaaaaagttactcaagcacaatcatcagtatccatgccagtgaggaaagcagtcaccctgaactgcctgtatgaaacaagttggtggtcatattatattttttggtacaagcaacttcccagcaaagagatgattttccttattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggggtctacgcgcactctcttactgggggataccggggaggcgccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatccttgtattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctaataattccgatccggctgctaa

SEQ ID NO: 7 - DNA, 3111 bp, Artificial sequence
  pGMT7
gatctcgatcccgcgaaattaatacgactcactatagggagaccacaacggtttccctctagaaataattttgtttaactttaagaaggagatatacatatgggatccatggtaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 8 - protein, 242 aa, Artificial sequence
  Amino acid sequence of  the gamma chain of the soluble DiS linked W gamma-delta TCR
MAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVTELGKKIKVFGPGTKLIITDKQLDADVSPKPTIFLPSIAETKLQKAGTYLCLLEKFFPDVIKIHWQEKKSNTILGSQECNTMKTNDTYMKFSWLTVPEESLDKEHRCIVRHENNKNGVDQEIIFPPIKTDVITMDPKDN

SEQ ID NO: 9 - protein, 233 aa, Artificial sequence
  Amino acid sequence of the delta chain of the soluble DiS linked W gamma-delta TCR
MQKVTQAQSSVSMPVRKAVTLNCLYETSWWSYYIFWYKQLPSKEMIFLIRQGSDEQNAKSGRYSVNFKKAAKSVALTISALQLEDSAKYFCALGVYAHSLTGGYRGGADKLIFGKGTRVTVEPRSQPHTKPSVFVMKNGTNVACLVKEFYPKDIRINLVSSKKITEFDPCIVISPSGKYNAVKLGKYEDSNSVTCSVQHDNKTVHSTDFEVKTDSTDHVKPKETENTKQPSKS

SEQ ID NO: 10 - DNA, 737 bp, Artificial sequence
  DNA encoding the TCR gamma chain of the soluble W    TCR mutated at the codon corresponding to TRGV9 Residue 49
tatacataatggcaggtcatttagaacaacctcaaatttccagtactaaaacgctgtcaaaaacagcccgcctggaatgtgtggtgtctggaataacaatttctgcaacatctgtatattggtatcgagagagacctggtgaatgcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggttacagagttgggcaaaaaaatcaaggtatttggtcccggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaagagtcactggacaaagaacatagatgtatcgtcagacatgagaataataaaaacggagttgatcaagaaattatctttcctccaataaagacagatgtcatcacaatggatcccaaagacaattaa

SEQ ID NO: 11 - DNA, 709 bp, Artificial sequence
  DNA encoding the TCR delta chain of the soluble W    TCR mutated at the codon in the FGXG motif of TRDJ1
tatacatatgcaaaaagttactcaagcacaatcatcagtatccatgccagtgaggaaagcagtcaccctgaactgcctgtatgaaacaagttggtggtcatattatattttttggtacaagcaacttcccagcaaagagatgattttccttattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggggtctacgcgcactctcttactgggggataccggggaggcgccgataaactcatctttggatgcggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctaa

SEQ ID NO: 12 - protein, 242 aa, Artificial sequence
  Amino acid sequence of a TCR gamma chain of the soluble W    TCR mutated at the codon corresponding to TRGV9 Residue 49
MAGHLEQPQISSTKTLSKTARLECVVSGITISATSVYWYRERPGECIQFLVSISYDGTVRKESGIPSGKFEVDRIPETSTSTLTIHNVEKQDIATYYCALWEVTELGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEESLDKEHRCIVRHENNKNGVDQEIIFPPIKTDVITMDPKDN

SEQ ID NO: 13 - protein, 233 aa, Artificial sequence
  Amino acid sequence of a TCR delta chain of the soluble W    TCR mutated at the codon in the FGXG motif of TRDJ1
MQKVTQAQSSVSMPVRKAVTLNCLYETSWWSYYIFWYKQLPSKEMIFLIRQGSDEQNAKSGRYSVNFKKAAKSVALTISALQLEDSAKYFCALGVYAHSLTGGYRGGADKLIFGCGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 14 - DNA, 737 bp, Artificial sequence
  DNA encoding the TCR gamma chain of the soluble W    TCR mutated at the codon corresponding to TRGJP Residue 13
tatacataatggcaggtcatttagaacaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggttacagagttgggcaaaaaaatcaaggtatttggttgcggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggagttgatcaagaaattatctttcctccaataaagacagatgtcatcacaatggatcccaaagacaattaa

SEQ ID NO: 15 - DNA, 709 bp, Artificial sequence
  DNA encoding the TCR delta chain of the soluble W    TCR mutated at the codon corresponding to TRDV1 Residue 49
tatacatatgcaaaaagttactcaagcacaatcatcagtatccatgccagtgaggaaagcagtcaccctgaactgcctgtatgaaacaagttggtggtcatattatattttttggtacaagcaacttcccagcaaatgcatgattttccttattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggggtctacgcgcactctcttactgggggataccggggaggcgccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctaa

SEQ ID NO: 16 - protein, 242 aa, Artificial sequence
  Amino acid sequence of a TCR gamma chain of the soluble W  TCR mutated at TRGJP Residue 13
MAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVTELGKKIKVFGCGTKLIITDKQLDADVSPKPTIFLPSIAETKLQKAGTYLCLLEKFFPDVIKIHWQEKKSNTILGSQEGNTMKTNDTYMKFSWLTVPEESLDKEHRCIVRHENNKNGVDQEIIFPPIKTDVITMDPKDN

SEQ ID NO: 17 - protein, 233 aa, Artificial sequence
  Amino acid sequence of a TCR delta chain of the soluble W  TCR mutated at TRDV1 Residue 49
MQKVTQAQSSVSMPVRKAVTLNCLYETSWWSYYIFWYKQLPSKCMIFLIRQGSDEQNAKSGRYSVNFKKAAKSVALTISALQLEDSAKYFCALGVYAHSLTGGYRGGADKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 18 - DNA, 734 bp, Artificial sequence
  DNA encoding the TCR gamma chain of the soluble B TCR mutated at the codon corresponding to and TRGC1 Exon 1 residue 60
tatacataatggcaggtcatttagaacaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggcaaagttgggcaaaaaaatcaaggtatttggtcccggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggagtgcaacaccatgaagactaacgacacatacatgaaatttagctggttaacggtgccagaagagtcactggacaaagaacacagatgtatcgtcagacatgagaataataaaaacggagttgatcaagaaattatctttcctccaataaagacagatgtcatcacaatggatcccaaagacaattaa

SEQ ID NO: 19 - DNA, 700 bp, Artificial sequence
  DNA encoding the TCR delta chain of the soluble B TCR mutated at the codon corresponding to TRDC Exon 1 Residue 47.
tatacatatggctattgaattagttcctgaacaccaaacagtgcctgtgtcaatagggatccctgccaccctcaggtgctccatgaaaggagaagcgatcggtaactactatatcaactggtacaggaagacccaaggtaacacaatcactttcatataccgagaaaaggacatctatggccctggtttcaaagacaatttccaaggtgacattgatattgcaaagaacctggctgtacttaagatacttgcaccatcagagagagatgaagggtcttactactgtgcctgtgaccctgttacgggggatactaggggcaccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatccttgtattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctaa

SEQ ID NO: 20 - protein, 241 aa, Artificial sequence
  Amino acid sequence of a TCR gamma chain of the soluble B TCR mutated at TRGC1 Exon 1 residue 60
MAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAKLGKKIKVFGPGTKLIITDKQLDADVSPKPTIFLPSIAETKLQKAGTYLCLLEKFFPDVIKIHWQEKKSNTILGSQECNTMKTNDTYMKFSWLTVPEESLDKEHRCIVRHENNKNGVDQEIIFPPIKTDVITMDPKDN

SEQ ID NO: 21 - protein, 230 aa, Artificial sequence
  Amino acid sequence of a TCR delta chain of the soluble B TCR mutated at TRDC Exon 1 Residue 47
MAIELVPEHQTVPVSIGIPATLRCSMKGEAIGNYYINWYRKTQGNTITFIYREKDIYGPGFKDNFQGDIDIAKNLAVLKILAPSERDEGSYYCACDPVTGDTRGTDKLIFGKGTRVTVEPRSQPHTKPSVFVMKNGTNVACLVKEFYPKDIRINLVSSKKITEFDPCIVISPSGKYNAVKLGKYEDSNSVTCSVQHDNKTVHSTDFEVKTDSTDHVKPKETENTKQPSKS

SEQ ID NO: 22 - DNA, 734 bp, Artificial sequence
  DNA encoding the TCR gamma chain of the soluble B TCR mutated at the codon corresponding to TRGV9 Residue 49
tatacataatggcaggtcatttagaacaacctcaaatttccagtactaaaacgctgtcaaaaacagcccgcctggaatgtgtggtgtctggaataacaatttctgcaacatctgtatattggtatcgagagagacctggtgaatgcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggcaaagttgggcaaaaaaatcaaggtatttggtcccggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggagttgatcaagaaattatctttcctccaataaagacagatgtcatcacaatggatcccaaagacaattaa

SEQ ID NO: 23 - DNA, 700 bp, Artificial sequence
  DNA encoding the TCR delta chain of the soluble B TCR mutated at a codon in the TRDJ1 FGXG motif
tatacatatggctattgaattagttcctgaacaccaaacagtgcctgtgtcaatagggatccctgccaccctcaggtgctccatgaaaggagaagcgatcggtaactactatatcaactggtacaggaagacccaaggtaacacaatcactttcatataccgagaaaaggacatctatggccctggtttcaaagacaatttccaaggtgacattgatattgcaaagaacctggctgtacttaagatacttgcaccatcagagagagatgaagggtcttactactgtgcctgtgaccctgttacgggggatactaggggcaccgataaactcatctttggatgcggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctaa

SEQ ID NO: 24 - protein, 241 aa, Artificial sequence
  Amino acid sequence of a TCR gamma chain of the soluble B TCR mutated at TRGV9 Residue 49
MAGHLEQPQISSTKTLSKTARLECVVSGITISATSVYWYRERPGECIQFLVSISYDGTVRKESGIPSGKFEVDRIPETSTSTLTIHNVEKQDIATYYCALWEAKLGKKIKVFGPGTKLIITDKQLDADVSPKPTIFLPSIAETKLQKAGTYLCLLEKFFPDVIKIHWQEKKSNTILGSQEGNTMKTNDTYMKFSWLTVPEESLDKEHRCIVRHENNKNGVDQEIIFPPIKTDVITMDPKDN

SEQ ID NO: 25 - protein, 230 aa, Artificial sequence
  Amino acid sequence of a TCR delta chain of the soluble B TCR mutated in the TRDJ1 FGXG motif
MAIELVPEHQTVPVSIGIPATLRCSMKGEAIGNYYINWYRKTQGNTITFIYREKDIYGPGFKDNFQGDIDIAKNLAVLKILAPSERDEGSYYCACDPVTGDTRGTDKLIFGCGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 26 - DNA, 734 bp, Artificial sequence
  DNA encoding the TCR gamma chain of the soluble B TCR mutated at the codon corresponding to TRGJP Residue 13
tatacataatggcaggtcatttagaacaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggcaaagttgggcaaaaaaatcaaggtatttggttgcggaacaaagcttatcattacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggagttgatcaagaaattatctttcctccaataaagacagatgtcatcacaatggatcccaaagacaattaa

SEQ ID NO: 27 - DNA, 700 bp, Artificial sequence
  DNA encoding the TCR delta chain of the soluble B TCR mutated at TRDV2 Residue 49.
tatacatatggctattgaattagttcctgaacaccaaacagtgcctgtgtcaatagggatccctgccaccctcaggtgctccatgaaaggagaagcgatcggtaactactatatcaactggtacaggaagacccaaggtaactgcatcactttcatataccgagaaaaggacatctatggccctggtttcaaagacaatttccaaggtgacattgatattgcaaagaacctggctgtacttaagatacttgcaccatcagagagagatgaagggtcttactactgtgcctgtgaccctgttacgggggatactaggggcaccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctaa

SEQ ID NO: 28 - protein, 241 aa, Artificial sequence
  Amino acid sequence of a TCR gamma chain of the soluble B TCR mutated at TRGJP Residue 13
MAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAKLGKKIKVFGCGTKLIITDKQLDADVSPKPTIFLPSIAETKLQKAGTYLCLLEKFFPDVIKIHWQEKKSNTILGSQEGNTMKTNDTYMKFSWLTVPEESLDKEHRCIVRHENNKNGVDQEIIFPPIKTDVITMDPKDN

SEQ ID NO: 29 - protein, 230 aa, Artificial sequence [sequence omitted]